(19) United States
(12) Patent Application Publication
(10) Pub. No.: US 2005/0054118 A1
(43) Pub. Date: Mar. 10, 2005

(54) High throughput screening method

(76) Inventor: Stewart J. Lebrun, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/848,962

(22) Filed: May 19, 2004

Related U.S. Application Data
(63) Continuation-in-part of application No. 10/376,351, filed on Feb. 27, 2003.
(60) Provisional application No. 60/471,638, filed on May 19, 2003.
(60) Provisional application No. 60/361,424, filed on Feb. 27, 2002.

(51) Int. Cl. G01N 33/543

(52) U.S. Cl. 436/518

(57) ABSTRACT

A high throughput screening method is described which employs a PVDF substrate for protein immobilization.

### RELATED APPLICATIONS

This application claims priority under 35 U.S.C. § 119(e) to U.S. Provisional Application No. 60/471,638, filed May 19, 2003 and is a continuation-in-part of U.S. application Ser. No. 10/376,351, filed Feb. 27, 2003 which claims priority under 35 U.S.C. § 119(e) to U.S. Provisional Application No. 60/361,424, filed Feb. 27, 2002. All of the above applications are incorporated herein by reference.

### BACKGROUND OF THE INVENTION

1. Field of the Invention

Preferred aspects of the present invention are related to improved microarray technology of expressed proteins, particularly throughout the lifespan of a cell during the progression of a degenerative disease.

2. Description of the Related Art

It is not surprising that there has been a recent surge in interest in the development of protein microarrays for diagnostic applications [1-4], as protein and antibody microarrays have the potential to serve as valuable tools for drug development and diagnostics [5]. Particularly exciting work has been done on printed antibodies and capturing proteins that serve as clinical markers for cancer [6]. In fact, entire complex tissue arrays have been used to identify disease markers for prostate cancer [7], renal duct and regulatory proteins [8], and proteins of the normal placenta [9]. Limited work has been done on assays involving enzymatic activity in the two-dimensional microarray format [10].

It has been said that there is “no PCR for proteins.” This illustrates one of the major challenges for protein microarrays. While arrays can hold tens-of-thousands of features, traditional methods make it difficult to purify these individual proteins. Several groups have tried cell-free methods for the production of proteins [11,12]. However, the yield and expense of these methods can be prohibitory for many research laboratories. It is also possible to develop peptide arrays using synthetically produced peptides [13,14]. This concept has been extended so that mRNA and protein are fused, allowing mRNA based identification of specific proteins found to be positive in the assay [15]. Allergen arrays [16] and autoimmune disease arrays have also been developed. One group has developed a yeast Proteome library using recombinant type expression in a bacterial system [17]. In a preferred embodiment, a specific proteome library for RA was developed uisng a high-throughput recombinant protein expression system and robotically transferring the recombinant proteins to a microarray format.

### SUMMARY OF THE INVENTION

Preferred embodiments of the invention are directed to a method of obtaining a protein or antibody profile for an individual by the steps of:
(a) preparing a PVDF or other 3-dimensional substrate for protein immobilization, which includes a rigid support and a PVDF or other hydrophobic polymer layer attached to the rigid support, wherein the PVDF layer has a surface chemistry adapted to immobilize a protein sample and wherein the substrate is configured to allow immobilization of a plurality of samples on discrete addressable spots thereon;
(b) applying one or more protein-containing samples to the PVDF or other 3-dimensional substrate to form a microarray;
(c) incubating the microarray with a blocker;
(d) reacting the microarray with a primary antibody to form an antibody-antigen complex or a protein-protein complex;
(e) exposing the complex to a second antibody or other detector molecule, wherein the second antibody is a detection agent; and
(f) determining a level of the detection agent and in turn determining the protein or antibody profile for the individual.

In preferred embodiments, the protein-containing sample includes cells, cell-free extracts, purified proteins or recombinant proteins. Preferably, the protein-containing samples are applied at 4-15° C. Preferably, the reacting step is carried out at a temperature of about 37° C. In preferred embodiments, the protein-containing samples are applied in an amount of 10⁻⁹ to 10⁻¹² grams protein per spot.

Preferably, determining the level of the detection agent is accomplished using an internal standard. Preferably, determining the level of the detection agent is performed by using a calorimetric assay. Preferably, the secondary antibody is Anti-IgE-AP (alkaline phosphatase). Preferably, the level of the detection agent is determined using a developer which includes Nitro-Blue Tetrazolium Chloride (NBT), and 5-Bromo-4-Chloro-3′-Indolylphosphate I-Toluidine salt (BCIP). Preferably, phosphate-containing buffers and reagents are avoided. In preferred embodiments, an image based detection system such as a flatbed scanner is used to determine the level of the detection agent.

Preferably, the protein-containing sample is spotted onto the PVDF or other hydrophobic polymer layer while the membrane is in a dry state.

In preferred embodiments, the blocker includes 0.1-5% casein in buffer. Preferably, the buffer is TBS. Preferably, the primary antibody is serum from a human subject.

In preferred embodiments, the thickness of the PVDF or other hydrophobic polymer layer is about 50-1000 μm. In alternate preferred embodiment, the thickness of the PVDF or other hydrophobic polymer layer is more than 1000 μm. Preferably, the PVDF or other hydrophobic polymer layer is applied by lamination. In alternate preferred embodiments, the PVDF or other hydrophobic polymer layer is applied by a spray or spin coat.

Preferably, the rigid support is a silanated material, glass or plastic. In preferred embodiments, the rigid support is a glass or plastic microscope slide. In alternate preferred embodiments, the rigid support has a 3 dimensional surface that includes channels for sample processing. Preferably, the substrate also includes a bar code. Preferably, the substrate also includes a removable protective film.

In some preferred embodiments, the substrate includes a template attached to the rigid support, wherein the template divides the PVDF substrate into at least two distinct sections, each section being configured to allow immobilization of a plurality of protein samples, and wherein the template is adapted to allow application of different chemical reagents to the at least two distinct sections of the PVDF substrate.

Preferred embodiments of the invention are directed to a method of screening for an antigen including the steps of:
(a) preparing a PVDF or other 3-dimensional hydrophobic substrate for protein immobilization, which includes a rigid support and a PVDF or other hydrophobic polymer layer attached to the rigid support, wherein the PVDF or other hydrophobic polymer layer has a surface chemistry adapted to immobilize a protein sample and wherein the substrate is configured to allow immobilization of a plurality of samples on discrete addressable spots thereon;
(b) applying a first capture antibody to the PVDF or other hydrophobic polymer substrate to form a microarray;
(c) treating the microarray with a blocker;
(d) reacting the microarray with one or more capture proteins to form an antibody-antigen complex;
(e) reacting the microarray with a second capture antibody;
(f) reacting the microarray with a third antibody, wherein the third antibody is a detection agent; and
(g) determining a level of the detection agent and in turn determining the presence of the antigen.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1. Theoretical model of protein species (P_{N}) and mass of each species (P_{M}) as they might fluctuate from cell formation (tm), through cell death (tD). This is defined by equation 1 in the text. The f(x), or functional Proteome is a theoretical slice at a specific point in time.

FIG. 2. Example of a 2D-gel electrophoresis of a yeast proteome (Lebrun, S. J. and McLaughlin, C. M., 1997): The first dimension is isoelectric focusing (IEF), where the isoelectric point of proteins visualized ranges from 4.5 to 7. The molecular weight (Mr×10⁻³) is determined by SDS page electrophoresis. Specific proteins can be identified based on standardized maps or mass spectrophotometry.

FIG. 3. The proteome display screening protocol: (1) Cloning cDNA libraries into vectors containing 6×His (2) Selection the colonies of interest using the process of transformation, plating on appropriate agar-media, induction and colony blotting. (3) Growing and inducing positive colonies in the appropriate media then transfer to 384-well dish (4) The proteins were printed onto slides and assayed with selective antibodies of interest.

FIG. 4. Calibration series: All calibration series must be present and must not deviate beyond a pre-determined value. This provides internal control for printing error. Additionally, all signals are normalized against a calibration series to control for between chip developing/processing deviations.

FIG. 5. Schematic representation of the immunochemistry applications used to assay the recombinant library. Chemistry is used to detect (a) protein-antibody interactions, (b) antibody-protein interactions, and (c) protein-protein interactions.

FIG. 6. (a) Spotware software interface, previewing the microarray to be analyzed. Images are scanned with a false-color, 24-bite color setting at 16oo-dpi. (b) Zoomed portion of the microarray chip, isolating the area within which proteins have been spotted. (c) Zoomed portion of the microarray, isolating select spots.

FIG. 7. The method of quantification—In order to quantify results, a calibration series of the detecting antibody is first spotted with the Miragene system onto the desired substrate. A series of five spots is deposited onto the substrate, where every five spots has a defined mass of analyte present. The top image (a) schematically illustrates the possible amounts of analyte in each calibration spot (these range from a mass of 0-pg to 25-pg). Once the substrate is assayed, it is scanned using some quantification software. In this case, the above image, (b), is obtained from the “Spotware” software. Because the mass of the protein was calculated before spotting, it is then possible to create a calibration curve, and plot the average signal intensity as a function of protein mass. An example is the IgE calibration curve shown as image (c) of this figure. As the signal intensities of other proteins are determined, the mass of the unknown proteins can then be determined from the calibration curve. For example, if the calibration curve illustrated above is used, and the signal intensity of one protein is found to be 0.4, then the mass of the protein has to be ˜6-pg. Finally, the protein can be quantified by plotting the protein mass as a function of titer.

FIG. 8. The scanned image of various clones (numbered 700 to 1040) spotted with the “Spotbot Protein Microarrayer” onto two Ni²⁺ slides. The top image is the result of assaying with a 1:500 ratio of RA control pool:Blocker. The bottom image is the result of assaying with a 1:500 ratio of RA patient pool: Blocker. Both cases utilized a ratio of 1:1000 α-Human IgG-AP:PBS as the secondary (detecting) antibody. Any spots apparent in the bottom image and not the top denote possible disease markers. Any spots apparent in both images denote possible location markers for this system.

FIG. 9. The scanned image of the L35 expression clone lysates spotted onto ζ-grip™ slides (top). The corresponding plot illustrates the signal intensity variation using different primary antibodies (bottom). The signal using the anti-his primary antibody indicates the presence of the 6×His tag within the cell lysate. The signal using the rheumatoid arthritis (RA) patient pool and the lack of signal using the RA control pool indicates the finding of a possible disease marker for RA.

FIG. 10. The plotted data of each clone's mass (in pg), and corresponding standard error, extracted from the results shown in FIG. 8. The mass was calculated using the average of five repeats.

FIG. 11. Predictive value for five clones found to be positive from the RA Proteome chip. Two of the proteins had significant homology to NADH dehydrogenase, and had significant predictive values. The other three potential disease markers had homologies to proteins involved in mitochondrial protein synthesis. This includes the 24 kDa subunit of the mitochondrial complex 1, exon 7; the mitochondrial elongation factor 1 alpha 1; and, the large subunit of the mitochondrial ribosome, L35.

FIG. 12. FIG. 12A shows results of the SLE 1:100 dilution of the SLE patient/control patient serum, with the corresponding list of positive antigens. FIG. 12B shows quantified results of this same dilution.

FIG. 13. FIG. 13A shows an example of the capture assay. FIG. 13B shows the quantified results of this p53 assay.

FIG. 14. Model to explain lower sensitivity of assay using PBS. As shown below, the Alkaline phosphatase (AP) reaction converst a soluble BCIP/NPT reactant to Diformazan. The excess phosphate competes for binding to the phosphatase and inhibits this reaction.

FIG. 15. FIG. 15A shows normalized signal intensity as a function of observed IU for mold using PBS. FIG. 15B shows normalized signal intensity as a function of observed IU for mold using TBS.

FIG. 16. FIG. 16 shows the detection of IgE reactivity to mold allergens. The signal intensity is plotted against the amount of patient serum added.

### DETAILED DESCRIPTION OF THE PREFERRED EMBODIMENT

A broad definition of the cellular proteome is the integral of expressed proteins from cell formation (mitosis) to cell death (senescence, apoptosis, or necrosis), throughout environmental challenges and diseases. The entire cellular proteome changes with respect to protein species and number of proteins as a function of time. If the proper high-throughput and data handling capability is developed, it may ultimately be possible to model the cellular proteome by taking functional, qualitative, and quantitative inventory at various time points throughout a cell's life span. The entire Proteome could be modeled as follows:
Proteome=[fₘ(x)+f₁(x)+f₂(x) . . . +f_{D}(x)]/dx [1]
where,
D is cell death;
m is cell mitosis; and
f(x) represents the range and mass of proteins at a given time and point.

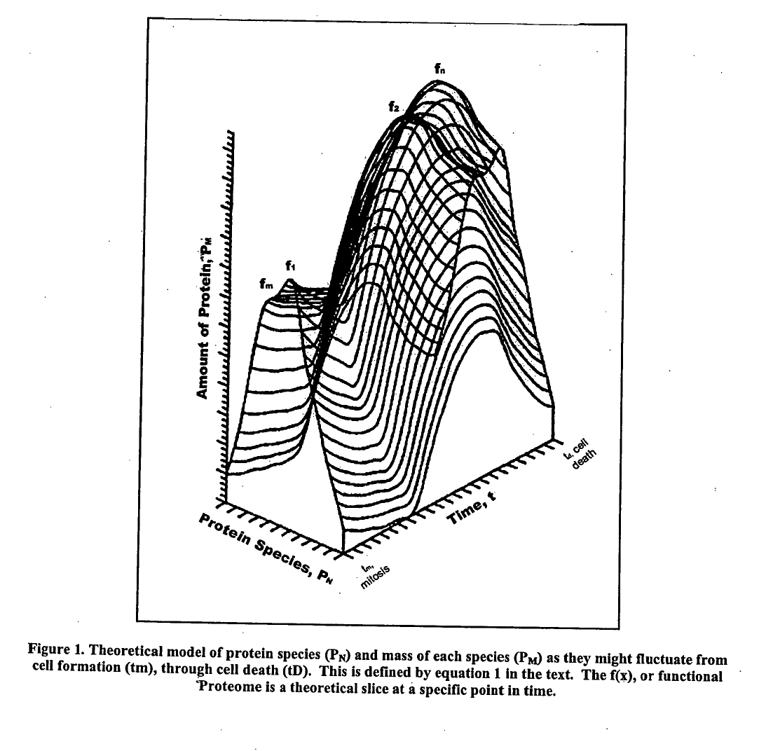

FIG. 1 provides a theoretical depiction of how this mathematical model can be graphically represented.

For each time point, f(x) has a range of different proteins (in the y-axis) of different masses (in the z-axis). Then, theoretically, the integral across all these time points describes the cell's life—from mitosis (fm(x)), through any disease or aberrant states (f(x)), to cell death (fd(x)). The number and species of proteins provides a molecular profile and staging of developmental or disease states.

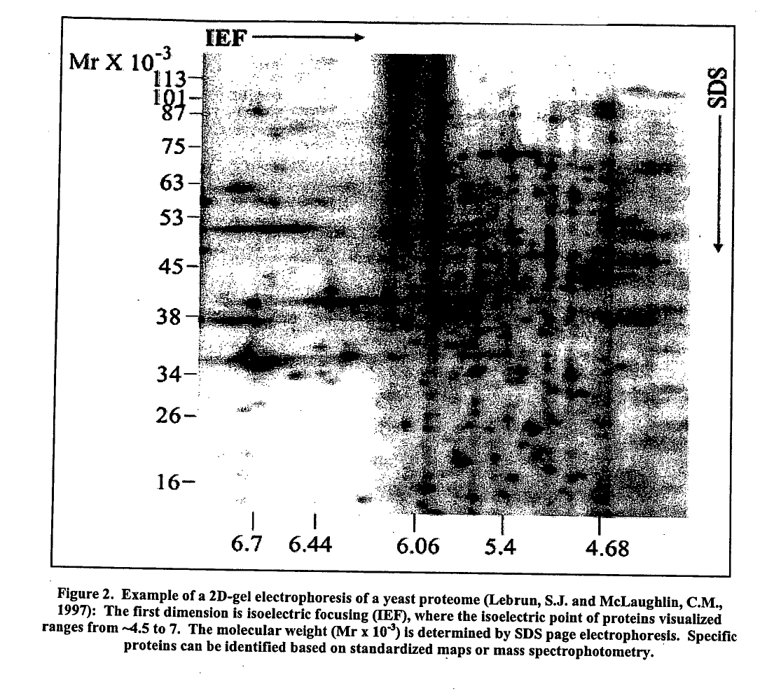

In a preferred embodiment, the Proteome was modeled to determine a given range of protein species associated with Rheumatoid Arthritis (hereafter referred to as RA). Relevant assays of such Proteome libraries can range from simple molecular patterns that define a diseased state, to complex analysis of cellular metabolism. Early work involved 2D-gel electrophoresis to image the Proteome and observe a “partial f(x),” as shown in FIG. 2.

Several thousand abundant proteins of the yeast cell line, XD83, can be seen, yet at any given time point, the typical yeast Proteome is around 30,000 proteins. Clearly, this approach is limited in the number of proteins, providing semi-quantitative or non-quantitative data, the fact that identification depends on mass spectrometry, and proteins are rendered inactive.

The raw number of samples that must be handled when studying the Proteome necessitates a technology that is miniaturiazed and automated. One such technology may be protein microarrays. Identification of the protein can be accomplished by tracing back to the cell line and the plasmid. The plasmid can then be sequenced, and the protein can be identified using genomic and proteomic databases. In addition, having clones that express the protein of interest provides an unlimited source of this protein for confirmation and other studies. While recombinant proteins do not always express or fold, are not modified, or otherwise have activity that is the same as in vivo proteins, it is reasoned that some recombinant proteins would have activity, and epitopes would remain intact.

Sample Prep

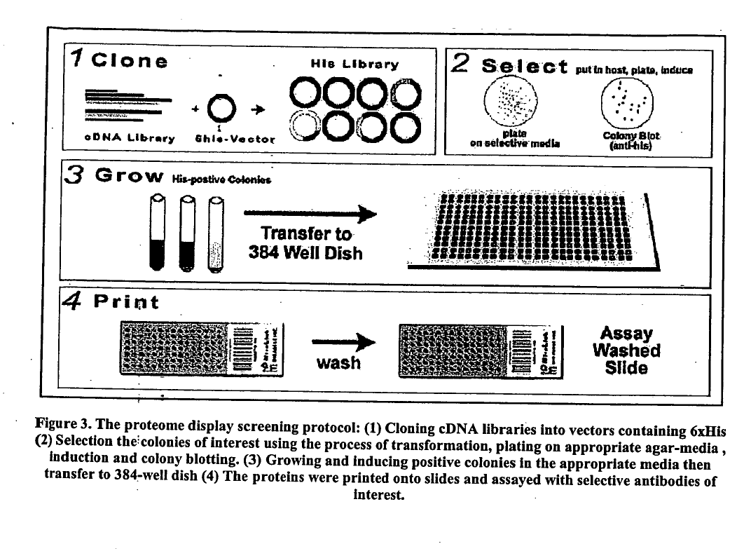

The term “functional proteome,” f(x)′, is defined as the variety and number of genes expressed in a given organism (or a given tissue) at a specific developmental or diseased state, that can be expressed as recombinant proteins. Two functional Proteome libraries (for human tissue types) are currently in development—one from a 64-yr old male kidney and the other from the synovium tissue, both derived from the cDNA library of six patients suffering from RA. FIG. 3 gives a schematic representation of the production and screening of clones used to produce a “Partial Functional Proteome” chip.

FIG. 3, box 1 illustrates a cDNA library and a vector tagged with six consecutive histidine residues (hereafter denoted as 6×his). The vector contains sequences involved in expression, purification, and identification of the recombinant proteins. This includes a leader sequence that causes proteins to be excreted, or cells that are lysed and pre-purified. The use of a leader sequence, which causes recombinant protein to be excreted into the media, reduces contamination of the printing system, and hence, lowers the potential of pin clogging. The vector also contains a specific antibody tag that allows the recombinant proteins to be assayed throughout the preparation. In this study, the 6×his peptide (which typically has low antigenicity) and a cMyc derived epitope (which has a much higher antigenicity) was used.

In the same image, the cDNA library is mass cloned into the 6×his vector to create a “6×his library.” This is done either by conventional enzymatic methods or amplification of the library by Taq polymerase-amplified PCR. The latter method results in the addition of single deoxyadenosine (A) to the 3′ ends of PCR products that can be cloned into the 6×his vector. The Proteome library is then introduced into bacterial cells and selected for uptake based upon its resistance to antibiotics, as shown in FIG. 3, box 2. Clones are pre-screened by replica plating and colony blotting using antibodies specific for the 6×his or cMyc epitope.

Colonies determined to be positive (based on antigenicity to anti-myc or anti-His antibodies) are transferred to growth medium and amplified. FIG. 3, box 3 shows the growth of specific recombinant clones. After amplification, the bacterium is induced to produce the protein, as this will increase the likelihood of the survival of clones containing a lethal gene or a gene that slows the growth of the bacterium. Then, cell lysates are separated by centrifugation, and media containing recombinant proteins is transferred to a 384-wells plate. In some cases, a 0.45-μm filtration step was performed. After induction, the lysate containing the recombinant protein and cells is separated by centrifugation. Theoretically, cells can also be grown directly in a 384-wells plate, hence avoiding fluidics transfer. If this is attempted, the z-axis of the robotic printing device must be adjusted accordingly to ensure that the pin does not penetrate the bacterial pellet at the bottom of the well.

The use of raw media with recombinant protein is not productive in applications where a large mass of protein is required, but is sufficient for the Proteome Display microarray, as it is sensitive in the pg to fg range (depending on specific assay).

Substrates

Microarray studies traditionally used derivitized glass slides with functional groups, such as poly-lysine, amine, and epoxide. One report suggests that ˜125-pg of human immunoglobulin is the detection cutoff on an optimized glass substrate [18]. This has low sensitivity compared to the “ζ-grip™” chip (Miragene, Inc. Santa Ana, Calif.), which can detect down to 2.5-pg of human immunoglobulin. The ζ-grip™ chip is essentially disclosed in U.S. application Ser. No. 10/376,351, filed Feb. 27, 2003, which is incorporated herein by reference. A summary of the ζ-grip™ and method of preparing follows.

PVDF was adhered to a glass support using an inert double-sided adhesive microfilm. Proteins (more specifically antigens) were spotted onto the dry surface of the PVDF and after drying were allowed to interact with antibodies with a conjugated secondary antibody. The opaque nature of the membrane together with the chemical detection system allows the interactions to be detected and analyzed on a low-cost flatbed scanner using light in the visual wavelength spectrum. The invention is expressly not limited to PVDF. Other hydrophobic polymers may also be used.

The protein microarray substrate can be used in a dry state to immobilize proteins. A hydrophobic membrane was included that immobilizes proteins in a reduced surface area with minimal diffusion across the membrane. The laminated membrane adheres to a glass surface with a double-sided inert adhesive microfilm, and preferably includes a protective polymer layer over the PVDF substrate surface.

The substrate can be used with multiple conjugated secondary antibodies such as Alkaline Phosphatase (AP), Biotin Protein A, or enzyme labels such as HRP or fluorescent dyes etc. In one preferred embodiment, the present invention optionally includes a barcode for test and/or sample identification and data archiving.

The described substrate provides a protein microarray with very little background noise. More specifically, the background noise for the ζ-GRIP™ PVDF-coated glass slide using the alkaline phosphatase (AP) reaction for detection of proteins, visualized using a conventional flatbed scanner, is less than about 100 lumens. More preferably, the background on the ζ-GRIP™ developed as above is between about 50 and 0 lumens. Most preferably, the background is from about 15 to 0 lumens. Similarly little to no background is seen when a fluorescent dye is used for protein detection on the ζ-GRIP™ PVDF-coated glass slide and imaged using a fluorescent scanner. In contrast, typical backgrounds seen using commercial protein substrates, e.g., slides with epoxy surface chemistries, are above 200 lumens and usually in the 300 to 400 lumen range.

Maximum signal intensities for the ζ-GRIP™ PVDF-coated glass slide in accordance with a preferred embodiment of the present invention using the Alkaline phosphatase reaction for detecting proteins and a conventional flatbed scanner for quantifying spot densities (otherwise referred to herein as “protein imaging”), analyzed using commercial imaging software (e.g., Adobe PHOTOSHOP®) are about 15,000 to 25,000 lumens. Maximum signal intensities for the Z-GRIP™ substrate using fluorescent detection chemistries and a fluorescent scanner are usually about 25,000 lumens. Moreover, in accordance with a preferred embodiment of the present invention, background for any detection chemistry on a PVDF-coated rigid support is less than about 1% of the maximal signal intensity, and more preferably, in the range of about 0.1% to about 1%, and most preferrably about 0.1% (e.g., 25 lumens background/25,000 lumens max signal).

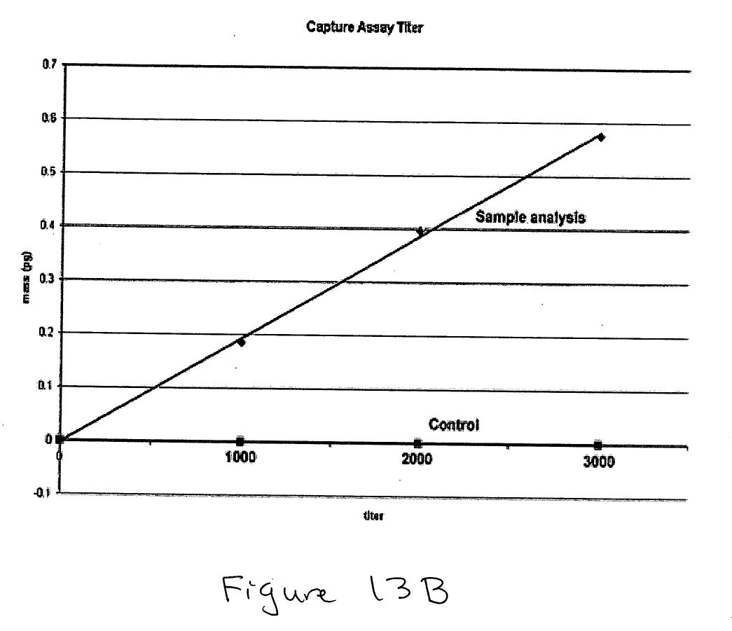

In addition to the advantages discussed above with regard to the higher signal-to-noise ratio seen with a preferred embodiment of the present invention, the ζ-GRIP™ PVDF-coated rigid supports also generate enhanced assay sensitivity because the hydrophobic PVDF surface facilitates superior protein spotting/density than the hydrophilic surface chemistries typically used for protein arrays (See e.g., Salinaro et al. WO 01/61042 which teaches the criticality of using a hydrophilic surface for biomolecular arrays). As a result of the hydrophobic nature of PVDF, protein samples spotted onto the PVDF surface tend to stay in high density, very discrete micro-spots (See magnified spots shown in FIG. 12), which do not spread and diffuse through the polymeric substrate. Thus, the protein density is relatively high compared to proteins spotted onto hydrophilic substrates. As a result of the high density, the concentration of protein does not become limiting on the subsequent detection reactions (e.g., labeled secondary antibody binding). Where protein spots have spread in hydrophilic substrates, the relative protein concentrations are much lower and become limiting on the detection reactions. Consequently, the sensitivity seen using the ζ-GRIP™ hydrophobic surface chemistry was observed to be approximately 1000 -fold greater than sensitivities obtained with the same proteins and detection reactions on a hydrophilic surface.

In one embodiment, the present invention provides a protein microarray with the capacity to immobilize up to 20,000 proteins in the open array format.

The term “immobilize,” and its derivatives, as used herein refers to the attachment of a bioactive species directly to a support member or to a support member through at least one intermediate component. As used herein, the term “attach” and its derivatives refer to adsorption, such as, physisorption or chemisorption, ligand/receptor interaction, covalent bonding, hydrogen bonding, or ionic bonding of a polymeric substance or a bioactive species to a support member. Although the substrate chemistries of the present invention are adapted to immobilize any proteins, peptides, or polypeptides, in some embodiments of the invention, protein antigens are disclosed as being immobilized. Accordingly, the terms “antigens” and “proteins” are used interchangeably throughout the disclosure unless explicitly otherwise indicated.

Materials:

Protein-immobilizing polymer: commercially available PVDF sheets or membranes. PVDF pellets may also be used in some modes of the invention.

Solid substrate: glass slides, plastic or other flat surfaced material. Optionally, the solid substrate may be silanated.

Adhesion material: Adhesive materials include commercially available double-sided adhesive film, silicon sealant, epoxy or other glue or suitable double sided tape, and direct chemical bonding.

In addition to an open format, another preferred embodiment of the present invention can be used with an attached template to provide multiple wells or sub arrays so that separate chemistries can be performed on the same slide. The number of individual wells could be 2 to several hundred.

One application is the separation of replicate arrays from each other on the same slide to allow patient comparisons or titrations. One or more steps can be performed in the small well. Then washing and other steps can be performed with larger volumes of solution across the whole slide.

In one embodiment, the present invention provides a three dimensional porous membrane attached to a solid support such as glass with an inert polymer. The three dimensional substrate captures and protects proteins in the porous membrane. The porous membrane has a thickness of approximately 150-500 μm, preferably 150-250 μm. The pore size is any pore size conventionally used for biological materials, particularly peptides and polypeptides. Typically, a pore size of 0.2 or 0.45 μm is used, preferably 0.45 μm. Note that these pore sizes refer to maximum pore size and that there may be a range of smaller pores, below the cutoff value, present on the membrane. These characteristics help maintain the morphology of the proteins. Proteins spotted onto the substrate surface maintain their integrity, providing increased sensitivity and assay consistency.

In one embodiment, the array substrate (Z-GRIP™) is assembled by hand on the laboratory bench. Under clean conditions the protective coating on one side of an inert double-side adhesive film is removed and attached to a solid support such as a glass slide. A sheet of PVDF is placed on the laboratory bench face down with the protective cover still in place. The remaining protective cover on the adhesive film is removed and the solid support is then pressed firmly onto the sheet of PVDF and allowed to dry. Using a sharp instrument, e.g., a razor blade, exacto knife etc., the PVDF membrane is trimmed to the size of the solid support. As an alternative to an inert double-sided adhesive film, other adhesive materials such as silicone, glue or double-sided tape can be used.

In a preferred embodiment, the Z-GRIP™ protein array is manufactured automatically under clean conditions. A large roll (approximately 1100 inches in length and 11 inches wide) of PVDF (obtained from Millipore Corporation) mounted on a 3.25-inch core is attached to a cutting and lamination machine. The machine automatically laminates a protective film to the upper side of the PVDF and an inert double-sided adhesive film with extended liner to the backside and cuts the sheets into the preferred size for automatic placement on 3″×1″ glass slides.

In another embodiment of the present invention, a layer of PVDF may be formed on a solid support by melting the polymer and applying it to the solid support. Modification of the PVDF chemistry is also deemed to fall within the scope of the present invention. Modifications may include carboxylation, amidization, and introduction of other reactive groups to the PVDF in order to promote immobilization of different bioactive species. In one embodiment, solid PVDF supports may be prepared by molding of the melted polymer.

Glass substrates give consistent spot deposition, however, immuno-reactivity has been found to be variable and less sensitive. Microarray substrates were originally developed for nucleotides, and so have been developed to aggressively bind nucleotides, such that they are linearized and available for hybridization. It is, therefore, surmised that aggressive covalent and ionic interactions between these substrates and proteins result in the deformation of epitopes, and may affect protein assays that are dependent on structure. Another issue with derivitized glass slides is its planarity. This reduces the amount of protein that can be bound to the substrate when compared to the amount that can be bound to a three-dimensional substrate. The amount of protein bound to the substrate directly determines signal intensity and signal to noise ratio. The above problems are avoided by the use of the ζ-grip™ substrate in the method as disclosed.

There has been a movement towards three-dimensional and hydrophilic substrates for protein microarrays. These are efforts to both increase the surface area and maintain the three-dimensional structure of the proteins [19]. However, it is not clear that hydrophilic substrates are acceptable for contact printing, as pins load by wick action. It is hypothesized that when protein solutions contact a hydrophilic substrate, the initial contacts result in a larger transfer of protein sample. This can result in uneven spot deposition, making quantification quite difficult.

The ζ-grip™ chip is a multi-layer membrane, which allows for increased loading, and may aid in the maintenance of protein structure, due to its three-dimensionality. More importantly, the ζ-grip™ chip allows for spot reproducibility with contact printing and sensitivity that allows the assay to use semi-crude, not concentrated recombinant proteins.

Printing System

In one embodiment, the procedure described herein utilizes the Telechem Stealth Microspotting Pins, or SMP3 (specifically that with a 75-μm tip), in conjunction with the Telechem Stealth Printhead, or SPH48, to print protein microarrays. This system was originally developed to print nucleotides, so the application to proteins may be a cause for concern, especially with regards to pin clogging and washing protocols. When printing, the Stealth pin goes through a wash/dry cycle, and in some cases, a sonication cycle prior to loading the protein (in buffer) solution. Loading takes only a few seconds, which is enough time for the entire slit pin chamber to fill with protein solution. The protein solution is then robotically contact-printed onto glass preprint slides. This allows any inconsistency of droplet formation at the pin tip to be removed, resulting in a consistent meniscus formation at the pin tip (typically 1-nl in volume). The protein solution is then printed onto the ζ-grip™ substrates in repeats of five for each substrate (Repetition of the protein spots is one element of quality control and will be further discussed later).

The goal of a successful protein print run is to deliver uniform and consistent volume (and, thus, mass) aliquots of protein sample to the substrate. The successful delivery of proteins is dependent upon physical characteristics of the printhead, pins, and the substrate. Proteins are heterogeneous and prone to precipitation that can clog microarray print pins (all of which were developed for nucleotides, a more homogeneous type of polymer). Therefore, the Stealth pin must be quality controlled, where quality control involves specific washing regimes and inspection under a microscope, as detailed by the manufacturer. Under a microscope, damaged pins will typically be observed to have bent or uneven contact regions, where contaminated pins will typically be seen with obstructions that clog. Proteins used should be dissolved in PBS, and filtered down to 0.45-μm, so that the probability of clogging in the 75-μm bore diameter of the Stealth pin is reduced. The preferred method of quality control, however, is to observe glass substrates printed with PBS. The first and last slide in the print rack should show that every sample intended to be printed has been printed, there is no carry over, and the first and last slides have the same number and volume of spots. At the very least, the pre-print area should be inspected to ensure that all spots are present. It is also important to properly wash and inspect the pins between protein runs, because proteins can precipitate and otherwise clog the Stealth channel.

Proteins in solution are a good growth source for contaminating microbes. And, the fact that recombinant proteins have been derived from bacterial sources further increases the likelihood of sample-protein contamination. It is, therefore, critical to maintain as much of a sterile environment within the robotic microarray printer as possible. Equipment should be thoroughly cleaned with 70% ethanol after all print runs, especially those involving potentially contaminated protein samples. Additionally, printing proteins at 4 to 8° C. will reduce microbial growth and maintain protein integrity, as proteins denature, are prone to proteolysis, etc., at higher temperatures (specifically at 25 to 37° C.). Printing at 4 to 8° C. helps retain the primary, secondary, and tertiary structure of recombinant proteins. Recall that protein-protein interactions are dependent upon the molecule's three-dimensional shape. The application of recombinant proteins can interfere with proper protein folding, secondary modifications, and activity, creating a challenge for protein microarrays. However, proteins printed from the same 384-wells plate at room temperature and at 8° C. proved the superiority of printing at the lower temperature. Proteins printed at room temperature lost all activity after two print runs (about 16-hrs), and the proteins printed at 8° C. lost reactivity typically after eight print runs (about 64-hrs).

Sample mass is typically in the ng to fg range for preferred embodiments of the present invention and in the μg range for ELISA type assays (recall that protein samples are expensive and in short supply). This is critical also because the miniaturization increases sensitivity, and (along with the enzymatic signal) allows amplification for the assay to be conducted with crude lysate. Results indicate that colorimetric protein microarrays performed on ζ-grip™ are approximately 50 to 1,000 times more sensitive then traditional calorimetric (alkaline phosphatase, hereafter denoted as AP) ELISA.

Calibration Markers and Tandem Blanks

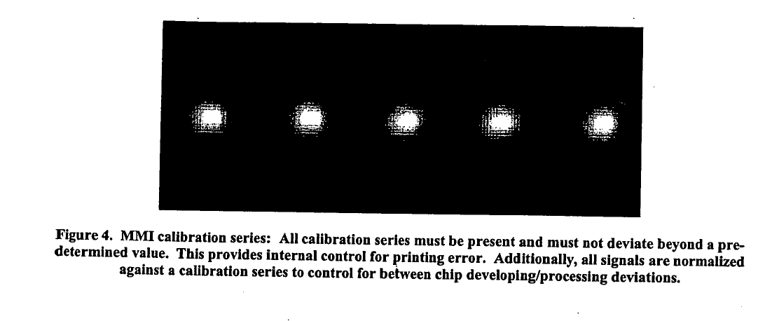

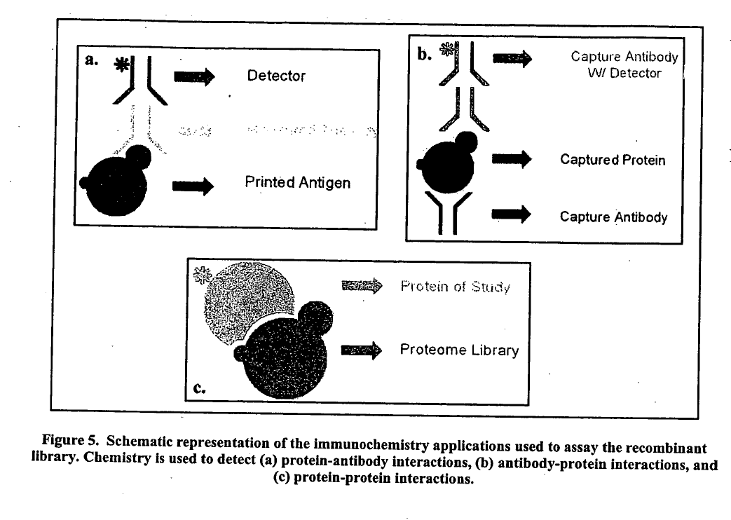

The print run can take from several minutes to several hours. It is suggested that one uses calibration markers and tandem blanks throughout the chip, and to place a glass slide in the first and last position of the printer. Calibration markers aid in the addressing of positive signals ensures that each array print was successful, and allows for user (and other) error that may affect spot intensity to be corrected (described later). The tandem blanks ensure that each chip had consistent printing without carryover. Typically calibration markers will be present on each array in repeats of five, throughout the array, in a pattern that allows addressing. Calibration markers are followed by PBS blanks to ensure that there is no sample carryover. Previous work using serial dilutions of some detector molecule has found that anti-human IgG or IgE conjugated alkaline phosphatase (AP) at a dilution of 1:100 works as a calibration marker (see FIG. 4).

Processing of the Chip

Chip processing for colorimetric detection takes from 1 to 3-hrs, and is described in detail in the Examples. Initially, the chips are blocked in ζ-grip™ blocking solution (0.1 to 5% casein in buffer), which has been found to be superior to the non-fat milk, BSA, or “blotto” type blockers. Blocking is a key determinant to the high signal-to-noise ratio, and can proceed from 20 to 60-min. A number of different ELISA type “sandwiches” can be used to assay the recombinant library, some examples of which are seen in FIG. 5.

The Proteome display chip has applications for finding protein-protein interactions, drug-protein interactions, and autoantibody assays. In these preferred embodiments, the Proteome library is printed onto the substrate, as previously described. The detection of these types of interactions can be accomplished in one of two ways—labeling the bait protein, or having a specific antibody directed against the bait protein and a secondary antibody containing the calorimetric enzyme (AP). Previous work has focused on finding autoantibodies (IgG or IgE) that may serve as diagnostic markers or clues to pathology. In a preferred embodiment, protocols as described in the Examples were used, in conjunction with clinically verified RA patient serum and sex/aged matched controls as the primary antibodies at a titer of 500. Initially, a pool of ten patient serum was used in order to enrich for autoantibodies that are widely occurring in the disease, and decrease concentration of individual specific autoantibodies. This first screen requires extensive verification and statistical validation using individual serum. Therefore, the same ten diagnosed RA patient serum (as determined by being positive to the Rheumatoid Factor) and ten-cohert control serum were assayed individually. From this, the predictive value of a given potential RA autoantigen was calculated, where

$$PredictiveValue = \frac{TP}{TP + FP} \qquad [ 2 ]$$

where,
   TP is the number of true positives
   FP is the number of false positives
      Scanning and Quantification

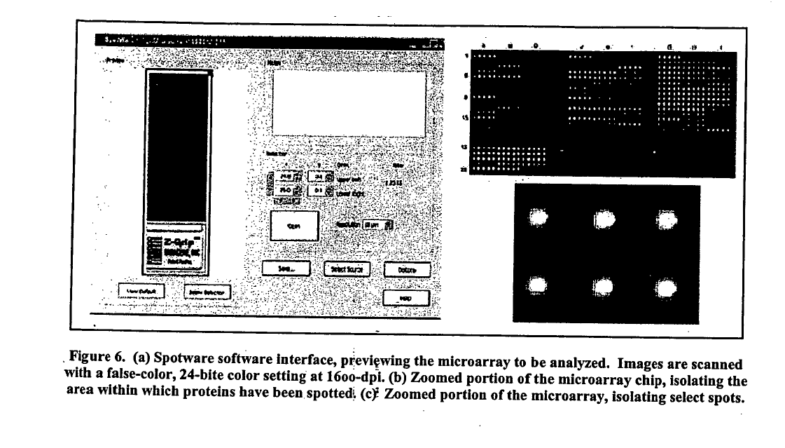

In a preferred embodiment, a colorimetric assay was used which differs from traditional fluorescent microarrays, in that it does not require a fluorometer [20] but still has good signal-to-noise properties. One of the advantages of the calorimetric system is the ability to use a low cost scanner with modified gain and custom software. This scanning system has been found to be sensitive, quantitative, and have low background. It also makes it reasonable for someone to order custom printed arrays, or print their own on a low cost printer, and do the detection for several thousand dollars (vs. 10 to 50 times this cost for a fluorescent or CCD imaging system). FIG. 6 shows the scanner software interface and some examples of scanned images.

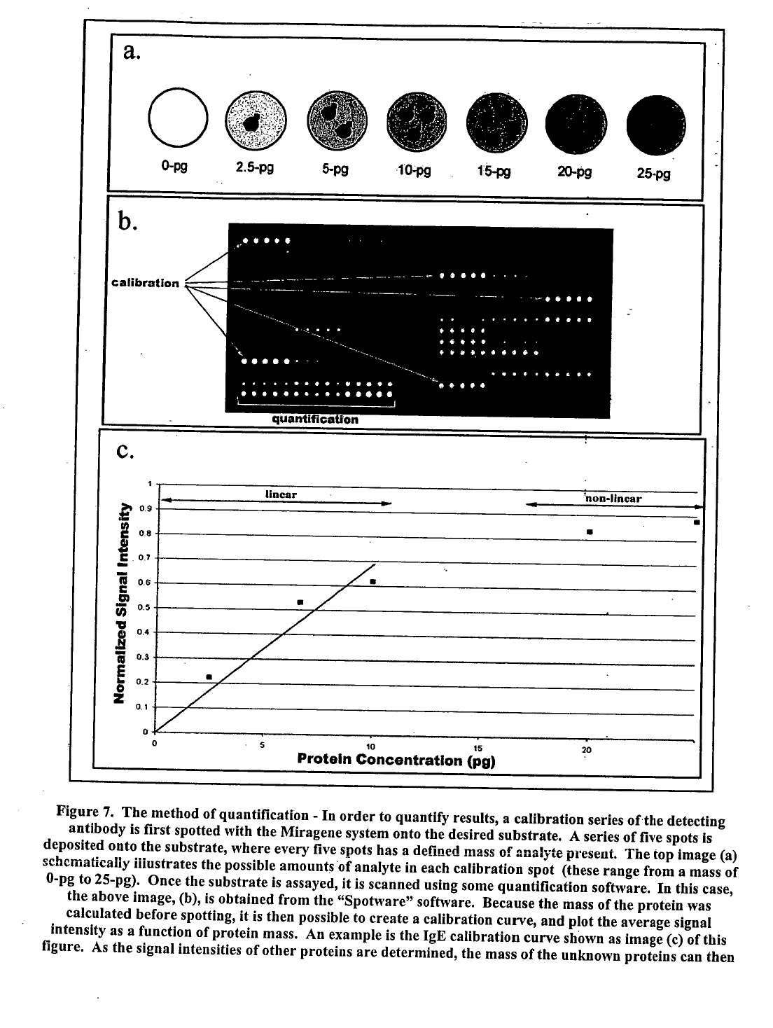

After scanning, images are transferred to some quantification software package (such as ArrayVision, ImageTool, or ScionImage). A pilot experiment determines the proper concentration of spotted reactant to dilution factor of unknown. The unknown in solution must be the limiting reagent for the assay to be quantitative. This is quite easily determined by a titration of unknown in solution and selection of dilution factor in the center of the positive slope, and not in the asymptotic region. We can define the dilution factor that gives quantitative results as the Qt (Quantitative Titer). This is illustrated in FIG. 7.

In one embodiment, quantification was accomplished by using internal standards and constructing a standard curve. Typically, these standards are the analyte in known mass. FIG. 7c is an example of a standard curve. The unknown signal intensity values were compared to standard curves to predict the mass of the unknown analyte.

Data Interpretation

Calibration markers are the first spots to be analyzed, as these ensure that the print run and development is successful. The successfulness of the print run and development is dependent upon the user, as they must pre-determine an acceptable variance (for example, 1 standard deviation or less). If successful, each spot (including the calibration marker) is given an average signal intensity value. Then, each value of the unknown samples is normalized to the value of the calibration marker, in order to correct for user (and other) error and interchip differences. A standard curve, similar to that shown in FIG. 7c is then constructed. In this example, the standard curve has a linear and nonlinear region, where the non-linear region cannot be used. Normalized unknown samples utilize this quantification plot to predict the mass of bound analyte. For example, in FIG. 7c, a normalized signal intensity of 0.4 indicates ˜6-pg of bound analyte. Protein microarrays performed on ζ-grip™ yield sensitivities in the pg to fg range.

Protein microarrays have wide application to disease diagnosis and staging. For example, protein microarrays have been used to profile serum proteins that define specific cancer types [21]. And in fact, laser desorption/ionization mass spectrometry type protein arrays have been used to identify the molecular weights of a number of RA markers from synovial fluid [22]. It has long been noted that patients with connective tissue diseases have increased levels of anti-mitochondrial antibodies. These typically have been assayed by Western Blotting of crude lysates of mitochondrial extracts [23]. In addition, muscle biopsy from patients with RA demonstrate mitochondrial abnormalities along with other histological changes such as degeneration and lipofuscin granules (a possible indication of necrosis) [24, 25]. Enzyme linked immuno-absorbant assay (ELISA) has shown RA patient sera has increased reactivity to crude mitochondrial extracts [26]. A number of mitochondrial proteins have implicated in the process of apoptosis. It is observed that apoptosis and cell cycle activity is abnormal in RA patients [27]. In addition, it has been found that synovial fluid contains factors that can directly damage the cell and cytotoxicity [28].

It may be that the molecular profile of RA contains “patterns” of protein associated with premature aging as well as “patterns of proteins” that are also present in other connective tissue disorders, such as Sjogren's syndrome, Systemic Lupus Erythematosus, Primary Biliary Cirrhosis, etc. Our relatively small sampling of the RA Proteome (about 4, 000 recombinant proteins) indicates that a number of mitochondrial proteins are likely to be autoantigens in RA. Several converging theories of aging suggest that aberrant mitochondrial activity produces excessive free radical damage, cell injury, and death. It would be interesting to look at a RA organism throughout the course of the disease, and determine how the f(x) changes throughout degeneration. Mathematical modeling of such data might provide clues to the diagnosis and treatment of degenerate diseases such as RA. The work described here demonstrates some of the capabilities and limitations of the partial Proteome chip we have developed.

### EXAMPLES

### Example 1

General Procedures

Bonding of PVDF to Substrate

PVDF was bonded to a solid substrate by the following steps: a) apply silicon, glue or double sided tape to solid substrate in even thin layer, b) under clean conditions, place sheet on lab bench and apply solid substrate (glue side facing PVDF sheet) to vinyl fluoride sheet, and c) press firmly and allow drying. Using an sharp instrument, e.g., a razor blade, exacto knife, etc., cut sheet so that it is size of solid substrate. The resulting PVDF bonded to a solid substrate is referred to herein as a chip, a slide and/or Zeta-Grip™ chip membrane.

The chips should be inspected before use. Use powder free nitrile gloves when handling the chips so as not to introduce dust. Place the chip on top of a bright light. Defective chips contain bubbles or defects.

Hand Spotting

All samples to be spotted were kept on ice until needed. Samples were pipetted onto the Zeta-Grip™ chip membrane (1 μl), making sure to not press the pipette tip down onto the membrane surface. The samples were slowly expelled such that each sample transfers directly to the chip surface. The printed chips were dried at 4-8° C. in a 60-80% humidified environment for at least 2 hours or overnight. Allowing the spots to dry in a relatively high humidity environment assures even distribution of protein sample within the printed spot. Any remaining “non-evaporated” sample observed after the drying period should be allowed to evaporate at room temperature for ˜10-15 min.

Contact Spotting

The SpotBot® Protein Edition Personal Microarrayer and Stealth Spotting Pins were used according to the manufacturer's instructions (www.arrayit.com). Basically, the wash buffer reservoir was filled with 50% ethanol. This was connected to the wash water container and the peristaltic pump was activated. Each target antigen was aliquoted into individual wells of the 384 well dish.

ζ-Grip™ slides were fitted on the right side of the instrument, with 2 plain microscope slides in the pre-print area. When working with a new sample, it is suggested that the first and last slide of the print-run be a normal glass slide. After the print-run, these are inspected using a microscope.

The SpotBot software program used was SPOCLE Generator. The following settings were used: Factory Default Profile; pintype SMP3; pin configuration set to 1×1; partial microplate was used; total microplate count is 1; the spots per sample is 5.

The settings in microarray printing were: spot spacing was 300 um; subgrid dimension was Column 15×Row 15; print offset was lateral 3.0×Vertical 5.0; cleaning cycle was 20 wash; and the rest of the settings were kept as computer default. Printing steps were preformed according to detailed steps in the SpotBot manual.

Non-Contact Spotting

The Biodot Biojet dispensing system was used according to the manufacturer's instructions (www.biodot.com).

Assay for Detection of Antibodies to Target Proteins or antigens

After printing, ζ-Grip™ slides were individually placed into empty incubation dishes. 10 mL of Blocker in PBS (or TBS) was added to each incubation dish/ζ-Grip™ slide combination. The incubation dishes were placed on a shaker, and mixed at room temperature for 1 hour. A defined volume (e.g. 10 μL in our examples) of sample or control was pipetted into incubation dish/spotted ζ-Grip™ slide/Blocker PBS combination. Do not apply sample directly onto ζ-grip™ chip rather pipette re-pipette 10 times into area away from chip. Mixing continued for 1 hour on the ELISA shaker. The Blocker-PBS (or TBS)/sample/control solutions were discarded and the slides were washed by adding 10 mL PBS (or TBS). Mixing was carried out at room temperature for 10 minutes. The washing step was repeated twice.

10 mL of diluted PBS (or TBS) was added into each incubation dish. 1 μL of secondary antibody (e.g. goat anti-human IgG-AP) was added to each incubation dish/slide/wash solution combination. Mixing was carried out for 1 hour at room temperature.

The solutions were discarded and the slides were washed with 10 mL of PBS (or TBS). Mixing was carried out for 10 minutes at room temperature. The wash solution was discarded. The washing was repeated twice.

ζ-Developer was prepared from stock solutions of Nitro-Blue Tetrazolium Chloride (NBT) (Immunopure® NBT, Pierce #34035) (5% NBT in 70% Dimethylformamide (DMF) (Fisher #BP1160-500)) and 5-Bromo-4-Chloro-3′-Indolylphosphate I-Toluidine salt (BCIP) (Immunopure® BCIP, Pierce #34040) (5% BCIP in 100% Dimethylformamide (DMF) (Fisher #BP1160-500)). Working concentrations of NBTand BCIP were 0.03% in Alkaline Phosphatase (AP) Buffer (0.1M Tris-HCl pH 9.5 (Sigma #B-9754), 0.1M NaCl (Fisher #S271-3), and 0.05M MgCl (Fisher #M33-500)). 10 mL of ζ-developer was added to cover the slide and mixed at room temperature, for 3 to 15 minutes (as pre-determined). The developer was discarded. Tap water was added to cover each slide for 2 minutes to stop further color development. The slides were air-dried overnight or in a slide dryer. The slides were scanned using an appropriate software program.

### Example 2

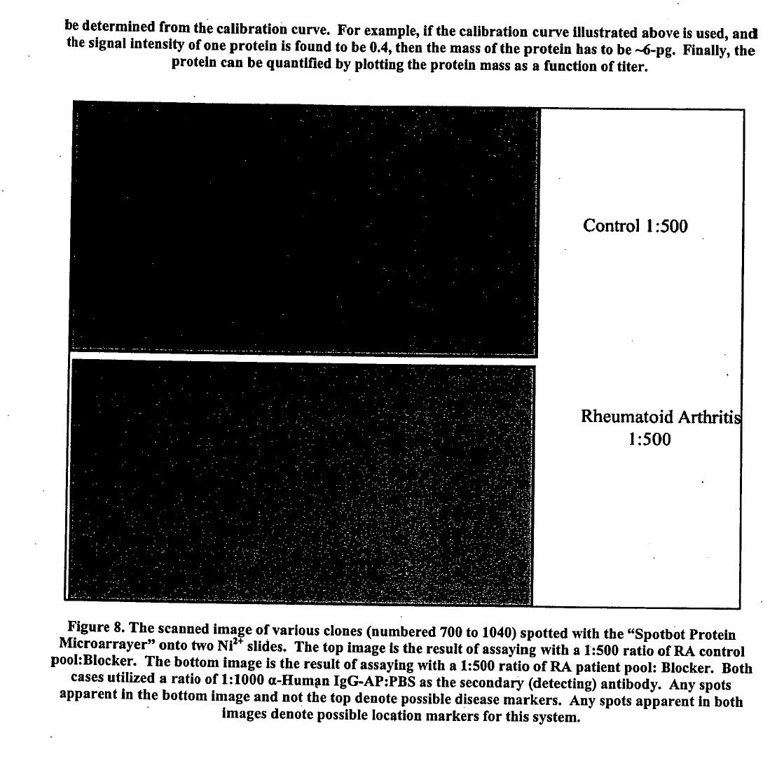

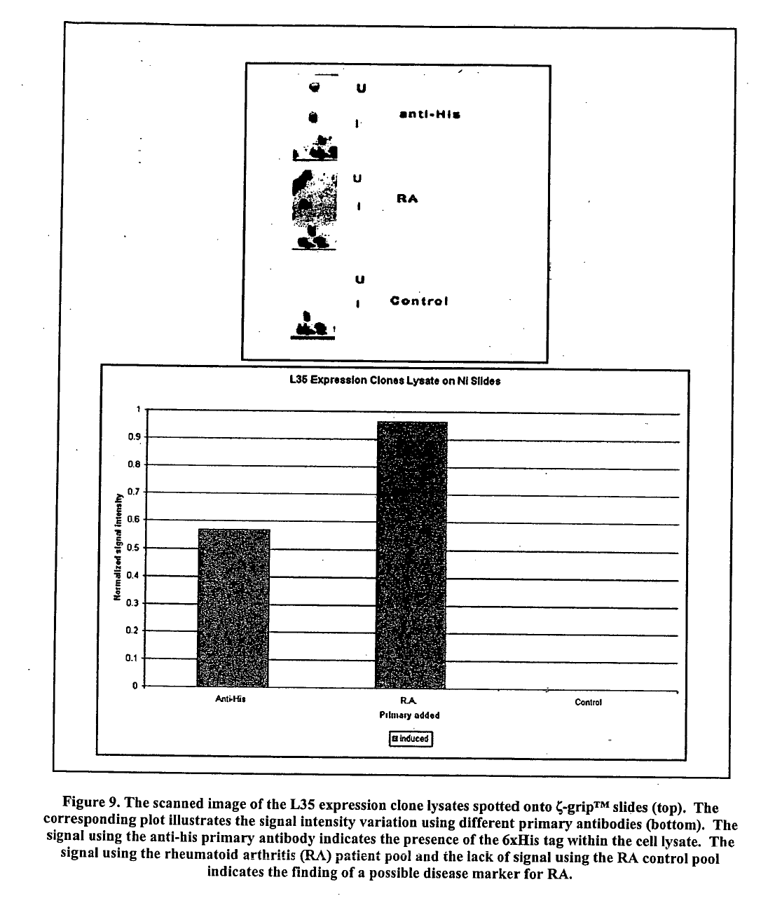

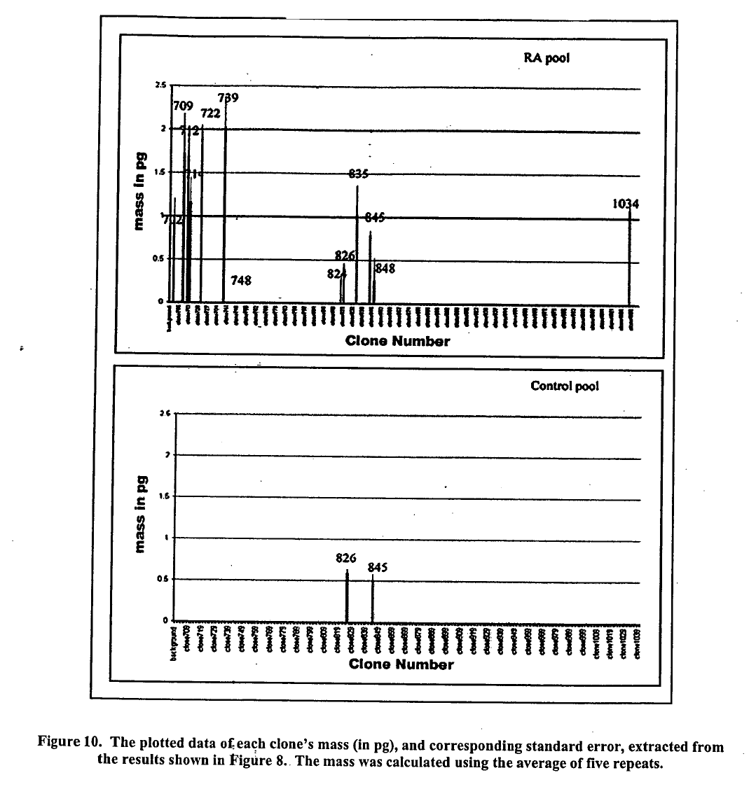

Experiments have been conducted using a Ni²⁺ substrate. FIG. 8 shows and example of this data.

There are a number of positive reactions for the RA patient pool at 1:500. There are also some corresponding positive reactions for control at 1:500. Several of these samples were traced back to clones. The clones were grown and used for plasmid prep and sequencing. From this early run, one of the positives that was present in RA but not control was a sequence with significant homology to the large subunit of the mitochondrial ribosome, L35. FIG. 9 shows raw data and quantified data for this potential RA marker.

FIG. 9a shows that a repeat assay of protein derived from this clone is positive for the anti-His. This indicates that it is likely to be expressed in the vector. The clone is also positive for the RA and negative for the control. FIG. 9b shows quantification of this, and the clear lack of signal in the control.

### Example 3

The next set of experiments described here utilized a leader sequence, and ζ-grip™ substrate. FIG. 10 shows a number of clones and the reactivity to pools of either RA patient serum, or control patient serum.

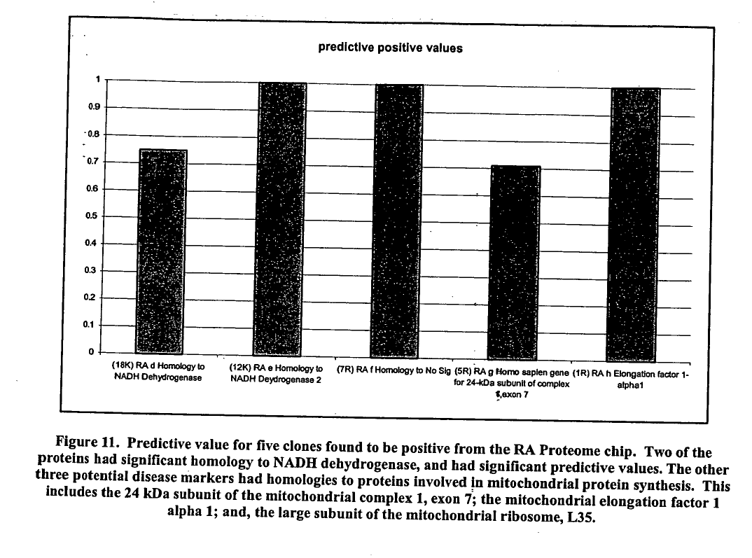

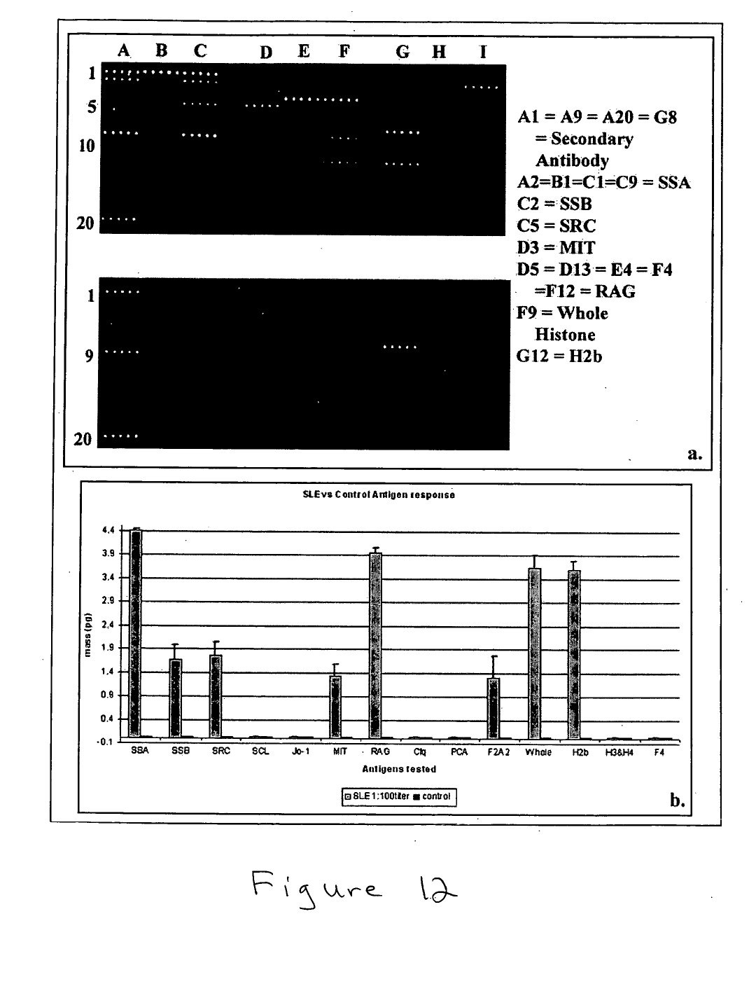

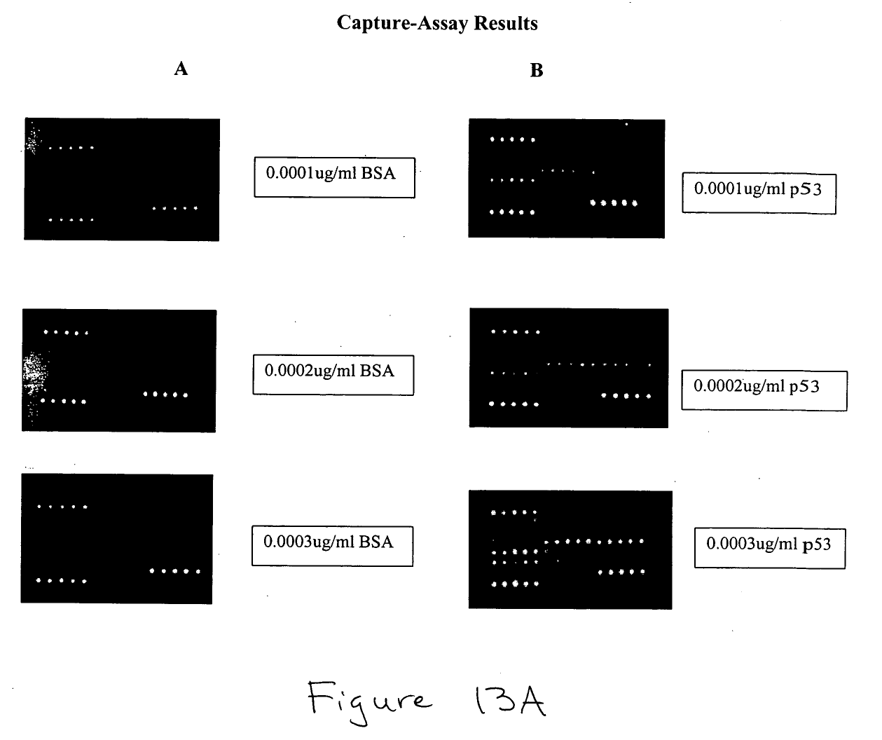

Four of the five RA recombinant proteins with significant predictive value had significant homology to mitochondrial proteins. However, these proteins, within the limits of our sequencing, appeared to have mutations and translocations. FIG. 11 shows the predictive value for five of these clones when assayed using individual serums.

Two different clones with significant homology to NADH dehydrogenase were found to have significant predictive value. It is interesting to note that the other dehydrogenase involved in mitochondrial energy production, pyruvate dehydrogenase has been previously identified as a disease marker for a number of rheumatic diseases, including RA [30]. Three of the potential disease markers had homologies to proteins involved in mitochondrial protein synthesis. This includes the 24-kDa subunit of the mitochondrial complex 1, exon 7; the mitochondrial elongation factor 1 alpha 1; and, as previously mentioned, the large subunit of the mitochondrial ribosome, L35. Assaying hundreds or thousands of individual disease markers for a given disease will provide a more complete molecular profile of the disease, and suggest and allow monitoring of therapeutics. We assayed approximately 4000 recombinant proteins, and found five with significant predictive value. Random sequencing of non-reactive clones did not indicate a bias in production of recombinant mitochondrial proteins. As can be seen in FIG. 11, sequences with homologies towards NaDH reductase, 24 kD subunit of the mitochondria, and the mitochondrial elongation factor 1 alpha all appear to give predictive values of RA over 70%. It is interesting to note that only 1 of the 5 is not related to the mitochondrial. This is a sequence with no significant homology. Therefore a total of 5 mitochondrial homologies were found to be significant (including L35), and only 1 other significant marker identified. It is interesting to note that current theories of age related degeneration and aging have implicated mitochondrial function as a key player in the aging process.

### Example 4

Autoantibody Protocol

Antigens were printed on ζ-grip™ chips. Patient serum containing measured antibody was incubated with the chip. After washing a secondary antibody containing Alkaline Phosphotase (AS) was incubated and allowed to bind to measure the antibody. After a final wash step, ζ-developer™ (Example 1) was added for a defined period of time. The reaction was stopped, the chip was dried and scanned in a colorimetric scanner and quantified using an appropriate quantification package.

The materials used for the autoantigen array of Table 1 are as follows:
Goat anti-human IgG-AP (Fishersci. Cat#P 131310)
Antigens:
   SSA (Immunovision cat#SSA-3000 lot#3448)
   SSB (Immunovision cat#SSB-3000 lot#2672)
   Jo-I (Immunovison cat#J01-3000 lot#3898)
   Scl-70 (Immunovision cat#SCL-3000 lot#3893)
   Smith (Immunovision cat#SMA-3000 lot#3589)
   Plasmid DNA (Immunovision cat#DNA-3000 lot#3422)
   SRC (Immunovision cat#SRC-3000 lot#3880)
   MIT (Immunovision cat#MIT-3000 lot#1365)
   PCA (Immunovision cat#PCA-3000 lot#3530)
   PAG (Immunovison cat#PAG-3000 lot#3717)
   Clq (Immunovision cat#CLQ-3000 lot)
   Histone subclass F2a2 (Immunovision cat#HIS-1012)
   Histone subclass F2b (Immunovision cat#HIS-1013)
   Histone subclass F3 and F2a1 (Immunovison cat#HIS-1014)
   Histone subclass F1 (Immunovision cat#HIS-1011)
   Whole Histone (Immunovision cat#HIS 1000)

Table 1 shows the array loading of the autoantigens into the 384 well plate.

TABLE 1
Diagram illustrating the location and type of antigens spotted on the ζ-Grip ™ slides for the example array.

| | A | B | C | D | E | F | G | H | I |
|---|---|---|---|---|---|---|---|---|---|
| 1 | Control +ve | SSA | SSA10/30 | SCL5/35 | SCL1/39 | | PCA1/39 | F2A2 his | F2A210/30 |
| 2 | SSA 5/35 | SSA1/39 | SSB | Jo-110/30 | Jo-15/35 | Jo-11/39 | F2A25/35 | F2A21/39 | whole his |
| 3 | SSB10/30 | SSB5/35 | SSB1/39 | MIT | MIT10/30 | MIT5/35 | whole10/30 | whole5/35 | whole1/39 |
| 4 | | SmA | SmA10/30 | MIT1/39 | RAG | RAG10/30 | | | |
| 5 | SmA5/35 | SmA1/39 | SRC | RAG5/35 | RAG1/39 | Clq | H2b | H2b10/30 | H2b5/35 |
| 6 | SRC10/30 | SRC5/35 | SRC1/39 | Clq10/30 | Clq5/35 | Clq1/39 | H2b1/39 | H3&H4 | H3&H410/30 |
| 7 | | SCL | SCL10/30 | PCA | PCA10/30 | PCA5/35 | H3&H45/35 | H3&H41/39 | F4 |
| 8 | SCL5/35 | SCL1/39 | Jo-1 | PCA1/39 | F2A2 | F2A210/30 | F410/30 | F45/35 | F41/39 |
| 9 | Control +ve | | SSA10/30 | F2A25/35 | F2A21/39 | Whole his | Control +ve | | |
| 10 | SSA 5/35 | SSA1/39 | | Jo-110/30 | Jo-15/35 | Jo-11/39 | whole10/30 | whole5/35 | whole1/39 |
| 11 | SSB10/30 | SSB5/35 | SSB1/39 | | MIT10/30 | MIT5/35 | H2b histone | H2b10/30 | H2b 5/35 |
| 12 | | | SmA10/30 | MIT1/39 | | RAG10/30 | H2b1/39 | H3&H4 | H3&H410/30 |
| 13 | SmA5/35 | SmA1/39 | | RAG5/35 | RAG1/39 | | H3&H410/30 | H3&H41/39 | F4 |
| 14 | SRC10/30 | SRC5/35 | SRC1/39 | Clq10/30 | Clq5/35 | Clq1/39 | F410/30 | F45/35 | F41/39 |
| 15 | | | SCL10/30 | | PCA10/30 | PCA5/35 | | | |
| 16 | | | | | | | | | |
| 17 | | | | | | | | | |
| 18 | | | | | | | | | |
| 19 | | | | | | | | | |
| 20 | Control +ve | | | | | | | | |
| “Control +ve” are positive controls used for calibration. The dilutions of the antigens are represented by numbers, where 10/30 indicates 10-μl of the original | | | | | | | | | |
| # antigen in 30-μl of PBS, 5/35 indicates 5-μl of the original antigen in 35-μl of PBS, and 1/39 indicates 1-μl of antigen in 39-μl of PBS. Bolded antigens indicate positive responses to | | | | | | | | | |
| # disease serum used in the experiments mentioned below. To be considered a positive response, each box/location will contain five positive reactions on the ζ-grip ™. | | | | | | | | | |

The slides were processed essentially as described in Example 1. ζ-Grip™ slides were individually placed into empty incubation dishes. 10 mL of Blocker (0.1-5% casein in PBS) was added to each incubation dish/ζ-Grip™ slide combination. The incubation dishes were loaded, placed on a shaker, and mixed at room temperature for 1 hour. A defined volume (10 μL) of sample or control (primary antibody) was micropipetted into the incubation dish/spotted ζ-Grip™ slide/Blocker PBS combination. Mixing was continued for 1 hour on the ELISA shaker.

The Blocker PBS/sample/control solutions were discarded and the slides were washed by adding 10 mL PBS and mixing at room temperature for 10 minutes. The washing step was repeated twice.

The wash solutions were discarded and 10 mL of diluted, PBS was added into each incubation dish. 1 μL of secondary antibody (e.g. goat anti-human IgG-AP) was added to each incubation dish/slide/wash solution combination and mixed for 1 hour at room temperature. The solutions from the incubation dishes were discarded, the slides were washed by adding 10 mL of PBS with mixing for 10 minutes at room temperature. The wash solutions were discarded. The wash step was repeated twice.

10 mL ζ-developer™ (Example 1) was added to cover the slide and incubated at room temperature, for 3 to 15 minutes. The developer was discarded. Tap water was added to cover each slide for 2 minutes to stop further color development. The slides were air-died overnight or span in a slide dryer. The slides were scanned and analyzed. The results are shown in FIG. 12.

FIG. 12 shows the results of the microarray of Table 1. The positive antigens are identified in FIG. 12A. Results of a pool of five Systemic Lupus Erythematosus (SLE) patients' serum, or a pool of five, corresponding age and sex matched control patients' serum. Positive responses were traced back to the original antigen in the 384-wells dish, as seen in Table 1. Positive control (Control+ve) for calibration is located at A1, A9, A20, and G9, and was used as a guide when the developing process was complete. All other antigens were diluted as pure, 10/30 (having a dilution of 10-μL original antigen in 30-μL Phosphate-buffered Saline (PBS)), 5/35 (having a dilution of 5-μL original antigen in 35-μL PBS), and 1/39 (having a dilution of 1-μl original antigen in 39-μl PBS). Pure Sjogren's Syndrome type A antigen (SSA) is positive at B1, where SSA 10/30 is positive at C1 and C9, and SSA 5/35 is positive at A2. Pure Sjogren Syndrome type B antigen (SSB) is positive at C2. The Smith/RNP complex antigen (SRC) is positive at C5, and the Mitochondial antigen (MIT) is positive at D3. As for the Recombination Activating protein (RAG), Pure RAG is positive at E4, RAG 10/30 is positive at F4 and F12, and RAG 5/35 is positive at D5 and D13. Autoantibodies against histones are also present in SLE patients. The positive histones are Whole histones at F9 and 12, F2A2 at H1, pure H2b at G12, H2b 10/30 at H12, and H2b 5/35 at I12. On the corresponding control slide, only Control+ve react.

### Example 5

Capture Assay Protocol

Specific antibodies (capture antibodies) were printed on ζ-grip™ chip. Sample containing protein analyte (capture protein) was incubated with the chip. After washing, a secondary capture antibody was added. Then a third antibody was added that recognizes the second antibody which was labeled with Alkaline Phosphotase (AP). Note this is one possible sandwich. One can also pre-treat the secondary and tertiary antibody and use this complex for detection or alternatively, the secondary antibody can be conjugated with AP. After a final wash step, ζ-developer™ was added for a defined period of time. The reaction was stopped, the chip was dried and scanned in a colorimetric scanner and quantified using an appropriate quantification package.

The following materials were used:
Purified protein p53 (Santa Cruz Biotechnology, cat#sc4246)
Rabbit polyclonal anti-p53 IgG (Santa Cruz Biotechnology, cat#sc6243)
Goat anti-rabbit IgG-AP (Santa Cruz Biotechnology, cat#sc2007)

Mouse monoclonal IgG (Santa Cruz Biotechnology, cat#scl26)

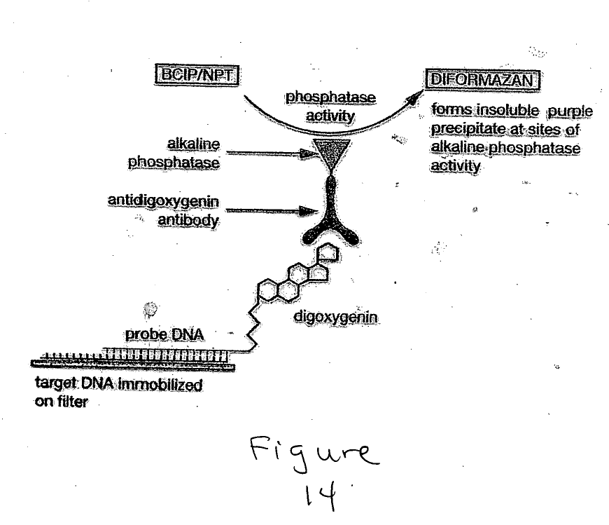

TABLE 2
Loading of the 384 well plate. The map corresponds to the
picture results for FIG. 13B.

| | | | |
|---|---|---|---|
| 1 | p53 (I/50 PBS) | p53 (I A1/50 PBS) | p53 (I A2/50 PBS) |
| 2 | p53 (I A3/50 PBS) | | BSA (I/50 PBS) |
| 3 | BSA (I A5/50 PBS) | BSA (I A6/50 PBS) | BSA (I A7/50 PBS) |
| 4 | | mouse mono p53 (50) | mm p53 (25/50 |
| | | | PBS) |
| 5 | mm p53 (10/40 PBS) | mm p53 (1/49 PBS) | |
| 6 | abcAM (50) | abcAM (25/50 PBS) | abcAM (10/40 PBS) |
| 7 | abcAM (1/49 PBS) | | anti-rabbit AP |
| 8 | anti-rabbit AP | | |

ζ-Grip™ slides were individually placed into empty incubation dishes. 10 mL of ζ-blocker™ (Example 1) in PBS was added to each incubation dish/ζ-Grip™ slide combination. Incubation dishes were placed on a shaker, and rotated at room temperature for 1 hour. Solutions containing proteins to be captured (p53) were pipetted into the ζ-Grip slide/Blocker combination. Mixing was carried out for 1 hour on the ELISA shaker.

The Blocker sample solutions were discarded and the slides were washed by adding 10 mL of PBS and mixing at room temperature for 10 minutes. The wash step was repeated twice.

Wash solutions were discarded. 10 mL of PBS was added into each incubation dish. Secondary antibody (for example: rabbit polyclonal IgG) was added into each incubation dish/slide/wash solution combination and mixed for 1 hour at room temperature.

The solutions were discarded from the incubation dishes, and the slides were washed by adding 10 mL of PBS and mixing for 10 minutes at room temperature. The wash step was repeated twice.

The wash solutions were discarded and 10 mL of diluted PBS was added into each incubation dish. The detector antibody (for example: anti-rabbit IgG-AP) was added into each incubation dish/slide/wash solution combination and incubated for 1 hour at room temperature.

The solutions from the incubation dishes were discarded. The slides were washed by adding 10 mL of PBS and mixing for 10 minutes at room temperature. The wash step was repeated twice.

The wash solutions were discarded. 10 mL of developer (Example 1) was added to cover the slide and mixed at room temperature, for 15 minutes, or whenever the first 5 dots appear.

The developer was discarded, and tap water was added to cover each slide for 2 minutes to stop further color development. The slides were air-dried overnight or spin dry.

The slides were scanned and the data was analyzed. Results are shown in FIGS. 13A and B. Chips were incubated with (A) BSA or (B) p53 at shown concentration. The three rows of 5 spots that appear in all the slides are calibration spots (positive controls).

### Example 6

Assay for Allergen-Responsive Human IgE

In another preferred embodiment, the sensitivity of the ELISA assays was combined with the multiplex advantages of microarray technology. The method uses PDVF membrane as described above in combination with an ELISA sandwhich and detection scheme. As little as 0.1 IU IgE can be detected using this enzyme-linked, colorimetric-based system. This method is well-suited to many applications including the screening of allergens where patients are typically screened for susceptibility to several hundred suspected allergens at one time.

Generation of Standard Curve:

The allergen extracts were aliquoted into a 384 well plate (5 ul/well) and robotically transferred to the zeta grip substrate using an array printer. The antigens were spotted based on equivalent activity values (determined using ELISA and World Health Organization standards) and in general the mass range was between 0.01-lpg/nL. A set of spots was included on each array for normalization and was pre-determined to be just below saturation. The normalization of spots corrects for chip-to-chip differences and maintains signal in a quantifiable region. If unknown signal is equal to or greater than the normalization signal, it is considered oversaturated and not quantifiable. The dynamic range of the assay is 3 orders of magnitude. All printed Zeta-Grip chips were labeled and individually placed into sterile plastic dishes, pre-filled with blocker (0.1-5% casein in buffer). Loaded dishes were then placed on an ELISA shaker (Titerplate Shaker, Labline Instruments, IL) and rotated (to allow for continuous mixing) at room temperature (25° C.) for 1 h. Next, diluted patient serum at desired titer (known generally as ‘primary antibody’) was added to each dish (with the slide and blocker) and continuously mixed for 1 hr. Solutions were appropriately discarded, and slides were washed 3 times by adding 10 mL of wash buffer (20-250 ul TBS). Anti-IgE-AP (alkaline phosphatase) was added for detection of patient IgE antibodies. Chips were washed as described above. After the last wash, 10 mL ζ-Developer (see Example 1) was added. This reagent is catalyzed by alkaline phosphatase and further developed as in a standard ELISA. The dishes were shaken for 1 h at room temperature. Solutions were discarded from the dishes, and slides were again washed three times. After discarding the last wash, 10 mL developer was added to the dish and shaken at room temperature for 15 min. The developer was discarded and distilled water was added to the dishes to stop further development for 2 minutes after which, Zeta-Grip chips were left to air-dry overnight. Developed chips were then scanned using the Miragene scanning system, where the resulting image is then quantified using commercial microarray software, such as ArrayVision™, Molecularware™, or TIGR™ (www.imagingresearch.com/products/ARV.asp, www.molecularware.com, www.tigr.org).

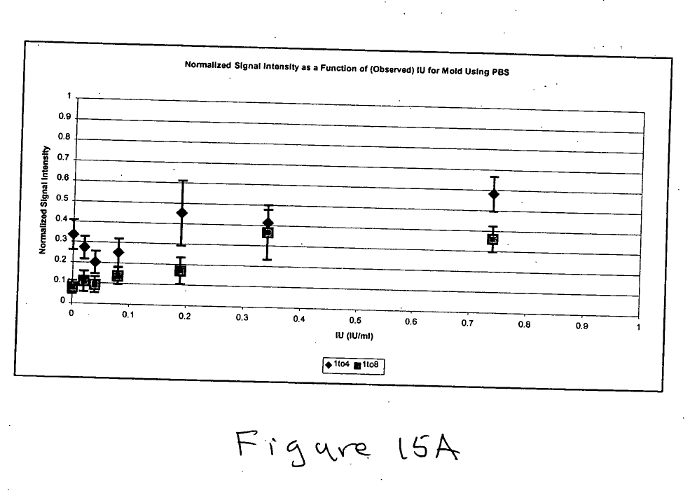

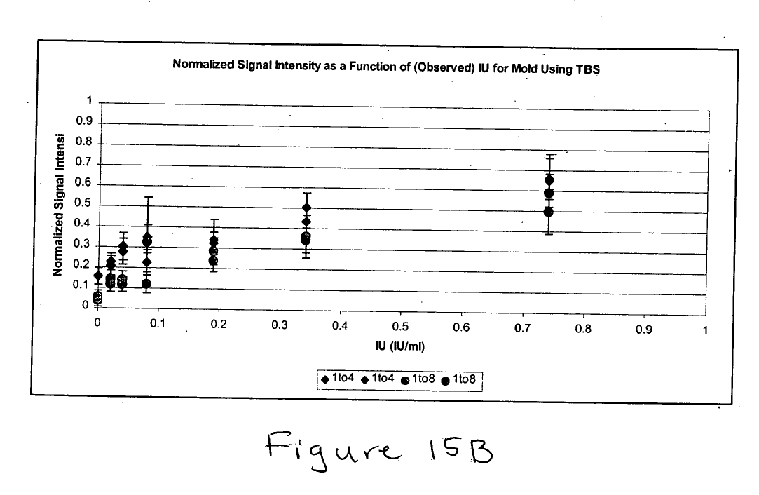

Note that in preferred embodiments, the buffer used is a TBS buffer. While other buffers may also be used, including PBS, improved sensitivity was achieved when a buffer which does not contain phosphate, such as TBS buffer, was used. Without being bound to any theory, it is hypothesized that the phosphate groups in PBS compete with the phosphate in the reactant for the AP assay. In preferred embodiments, buffers and other reagents containing phosphate groups are avoided when a phosphate-dependent assay, such as AP, is used as the enzyme in the enzyme-linked secondary antibody. A model illustrating this hypothesis is shown as FIG. 14. Activities are about 10 fold higher when TBS is used as the buffer compared to PBS. See FIGS. 15A and B.

FIG. 15 shows plotted data illustrating the normalized signal intensity as a funcion of IU for a single patient. Data focuses on the mold allergen, and plots the observed (vs. the expected) IU. Data points represent the average signal intensity PER CHIP along with their corresponding standard error PER CHIP. I.e., these data points all come from different chips, even though the spotted allergen and washing solution are the same. PBS indicates that the sample was assay-washed with phosphate-buffered saline (FIG. 15A). TBS indicates that the sample was assay-washed with Tris buffered saline. “1 to 4” and “1 to 8” indicates the dilution of the mold allergen.

While initial studies were done at room temperature, it was later determined that the assay could be further optimized by increasing the temperature to about 37° C. for the reaction between the antibody and the antigen. Further increased sensitivity compared to PBS was achieved by dilution of patient serum with equine horse serum (20-500 μl/10 ml was used). The amount of antibody was optimal at about 20 μl/10 ml for range. 250 μl/10 ml gave the best sensitivity, but saturated for high positive patients.

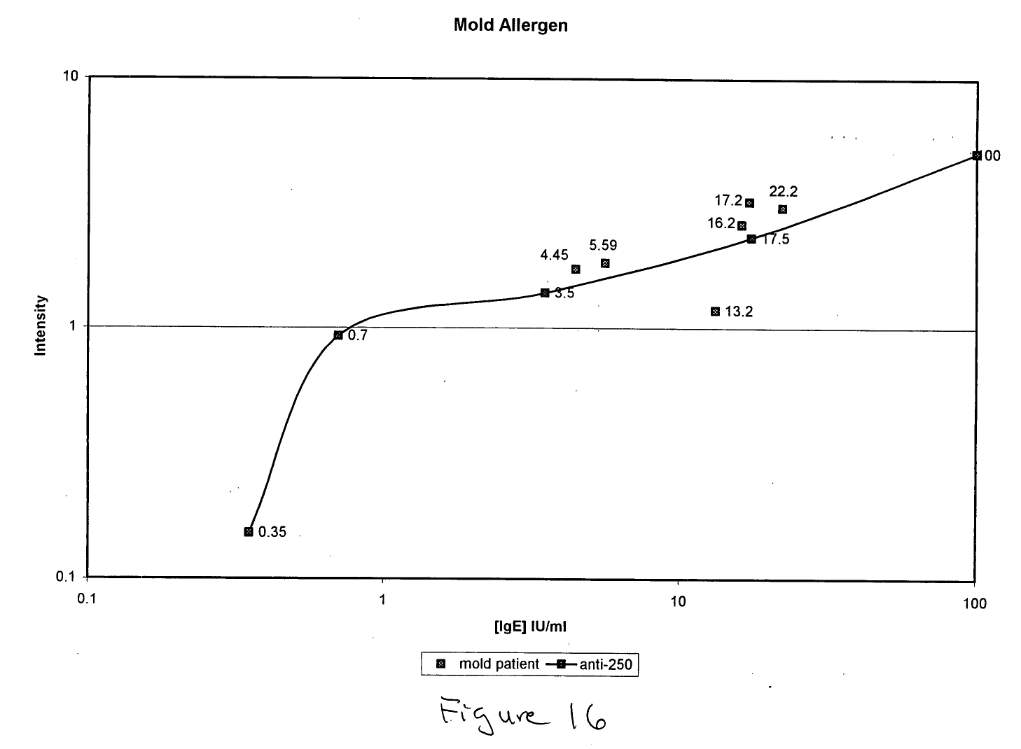

FIG. 16 shows the detection of IgE reactivity to spotted mold allergens by the colorimetric microarray assay described above, compared to a calibration curve from WHO standards. The assay has a sensitivity below the current WHO cutoff of 0.35 IU, making this assay as sensitive as non-microarray IgE tests. Coupled with the multiplexing advantages of microarray technology, our assay is a substantial improvement over these traditional in vitro tests. Additionally, the use of enzyme-linked detection adds a further improvement over other allergen arrays that rely on a lesser sensitive fluorescent detection scheme.

It will be understood by those of skill in the art that numerous and various modifications can be made without departing from the spirit of the present invention. Therefore, it should be clearly understood that the forms of the present invention are illustrative only and are not intended to limit the scope of the present invention.

### REFERENCES (Incorporated Herein by Reference)

1. Wilson, D. S. and S. Nock. Recent developments in protein microarray technology. Angew Chem Int Ed Engl 2003 Feb. 3;42(5):494-500.
2. Ng, J. H. and L. L. Ilag. Biomedical applications of protein chips. J Cell Mol Med 2002 July-September;6(3):329-40.
3. Eickhoff, H., Z. Konthur, A. Lueking, H. Lehrach, G. Walter, E. Nordhoff, L. Nyarsik and K. Bussow. Protein array technology: the tool to bridge genomics and proteomics. Adv Biochem Eng Biotechnol 2002;77:103-12.
4. Talapatra, A., R. Rouse and G. Hardiman. Protein microarrays: challenges and promises. Pharmacogenomics 2002 July;3(4):527-36.
5. Huels, C., S. Muellner, H. E. Meyer and D. J. Cahill. The impact of protein biochips and microarrays on the drug development process. Drug Discov Today 2002 Sep. 15;7(18 Suppl):S119-24.
6. Krieg, R. C., C. P. Paweletz, L. A. Liotta and E. F. Petricoin 3^{rd}. Clinical proteomics for cancer biomarker discovery and therapeutic targeting. Technol Cancer Res Treat 2002 August;1(4):263-72.
7. Zellweger, T., C. Ninck, M. Mirlacher, M. Annefeld, A. G. Glass, T. C. Gasser, M. J. Mihatsch, E. P. Gelmann and L. Bubendorf. Tissue microarray analysis reveals prognostic significance of syndecan-1 expression in prostate cancer. Prostate 2003 Apr. 1;55(1):20-9.
8. Legato, J., M. A. Knepper, R. A. Star and R. Mejia. Database for renal collecting duct regulatory and transporter proteins. Physiol Genomics 2003 Apr. 16; 13(2): 179-81.
9. Batorfi, J., B. Ye, S. C. Mok, I. Cseh, R. S. Berkowitz and V. Fulop. Protein profiling of complete mole and normal placenta using ProteinChip analysis on laser capture microdissected cells. Gynecol Oncol 2003 March;88(3):424-8.
10. Chen, G. Y., M. Uttamchandani, Q. Zhu, G. Wang and S. Q. Yao. Developing a Strategy for Activity-Based Detection of Enzymes a Protein Microarray. Chembiochem 2003 Apr. 4;4(4):336-339.
11. He, M. and M. J. Taussig. DiscernArray technology: a cell-free method for the generation protein arrays from PCR DNA. J Immunol Methods 2003 Mar 1;274(1-2):265-70.
12. Tabuchi, M., M. Hino, Y. Shinohara and Y. Baba. Cell-free protein synthesis on a microchip. Proteomics 2002 April;2(4):430-5.
13. Salisbury, C. M., D. J. Maly and J. A. Ellman. Peptide microarrays for the determination of protease substrate specificity. J Am Chem Soc 2002 Dec. 18;124(50):14868-70.
14. Reimer, U., U. Reineke and J. Schneider-Mergener. Peptide arrays: from macro to micro. Curr Opin Biotechnol 2002 August;13(4):315-20
15. Weng, S., K. Gu, P. W. Hammond, P. Lohse, C. Rise, R. W. Wagner, M. C. Wright and R. G. Kuimelis. Generating addressable protein microarraya with PROfusion colvalent mRNA-protein fusion technology. Proteomics 2002 January;2(1):48-57.
16. Harwanegg, C., S. Laffer, R. Hiller, M. W. Mueller, D. Kraft, S. Spitzauer and R. Valenta. Microarrayed recombinant allergens for dignosis of allergy. Clin Exp Allergy 2003 January;33(1):7-13.
17.
18. Seong, S. Y. Microimmunoassay using a protein chip: optimizing conditions for protein immobilization. Clin Diagn Lab Immunol 2002 July;9(4):927-30.
19. Groves, J. T. Membrane array technology for drug discovery. Curr Opin Drug Discov Devel 2002 July;5(4):606-12.
20. Wiese, R. Analysis of several fluorescent detector molecules for protein microarray use. Luminescence 2003 January-February;18(1):25-30.
21. Poon, T. C., T. T. Yip, A. T. Chan, C. Yip, V. Yip, T. S. Mok, C. C. Lee, T. Ieung, S. K. Ho and P. J. Johnson. Comprehensive proteomic profiling identifies serum proteomic signatures for detection of hepatocellular carcinoma and its subtypes. Clin Chem 2003 May;49(5):752-60.
22. Uchida, T., A. Fukawa, M. Uchida, K. Fujita and K. Saito. Application of a novel protein biochip technology for detection identification of rheumatoid arthritis biomarkers in synovial fluid. J. Proteome Res 2002 November-December;1(6):495-9.
23. Mouritsen, S., E. Demant, H. Permin and A. Wiik. High prevalence of anti-mitochondrial antibodies among patient with some well-defined connective tissue diseases. Clin Exp Immunol 1986 October;66(1):68-76.
24. Wroblewski, R. and R. Nordemar. Ultrastructural and histochemical studies of muscle in rheumatoid arthritis. Scand J Rheumatol 1975;4(4):197-204.
25. Corvetta, A., P. A. Ghirelli, P. J. Spaeth, F. Orecchioni, M. Galeazzi, G. Sprovie, R. Butler and U. E. Nydegger. An immunochemical study of the synovial fluid can contribute the distinction between rheumatoid and non rheumatoid arthritis. Clin Exp Rheumatol 1984 April-June;2(2):111-7.
26. Mouritsen, S., M. Hoier-Madsen, E. J. Demant and H. Permin. Enzyme-linked immunosorbent assay for determination of anti-mitochondrial antibodies. Acta Pathol Microbiol Immunol Scand [C] 1985 October;93(5):205-10.
27. Perlman, H., L. J. Pagliari and M. V. Volin. Regulation of apoptosis and cell cycle activity in rheumatoid arthritis. Curr Mol Med 2001 November;1(5):597-608.
28. Blann, A. D. An assay for estimating the cytotoxicity of synovial fluid from patients with rheumatoid arthritis. Scand J Clin Lab Invest 1985 November;45(7):665-7.
29. Gordon, R. D., J. A. Young, S. Rayner, R. W. Luke, M. L. Crowther, P. Wordswo, G. Hassall, J. Evans, S. A. Hinchliffe and et al. Purification and characterization of endogenous peptides extracted from HLA-DR isolated from the spleen of a patient with rheumatoid arthritis. Eur J Immunol 1995 May;25(5):1473-6.
30. Zurgil, N., R. Bakimer, H. M. Moutsopoulos, A. G. Tzioufas, P. Youinou, D. A. Isenberg, M. Scheinberg, T. Kveder, B. Rozman, C. Luderschmidt and et al. Antimitochondrial (pyruvate dehydrogenase) autoantibodies in autoimmune rheumatoid diseases. J Clin Immunol 1992 May;12(3):201-9.

1. A method of obtaining a protein or antibody profile for an individual comprising the steps of:
(a) preparing a PVDF or other 3-dimensional hydrophobic substrate for protein immobilization, comprising a rigid support and a PVDF or other hydrophobic polymer layer attached to said rigid support, wherein said PVDF layer has a surface chemistry adapted to immobilize a protein sample and wherein said substrate is configured to allow immobilization of a plurality of samples on discrete addressable spots thereon;
(b) applying one or more protein-containing samples to the PVDF or other 3-dimensional substrate to form a microarray;
(c) incubating the microarray with a blocker;
(d) reacting the microarray with a primary antibody to form an antibody-antigen complex or a protein-protein complex.
(e) exposing the complex to a second antibody or other detector molecule, wherein said second antibody or other detector is a detection agent; and
(f) determining a level of the detection agent and in turn determining the protein, or antibody profile for an individual.

2. The method of claim 1, further comprising using an image based detection system such as a flatbed scanner to determine the level of the detection agent.

3. The method of claim 1, wherein the protein-containing sample is selected from the group consisting of cells, cell-free extract, purified protein and recombinant protein.

4. The method of claim 1, wherein the protein-containing samples are applied at 4-15° C.

5. The method of claim 1, wherein the protein-containing samples are applied in an amount of 10⁻⁹ to 10⁻¹² grams protein per spot.

6. The method of claim 1, wherein determining the level of the detection agent is accomplished using an internal standard.

7. The method of claim 1, wherein determining the level of the detection agent is performed by using a colorimetric assay.

8. The method of claim 1, wherein the protein-containing sample is spotted onto the PVDF or other hydrophobic polymer layer, wherein the membrane is in a dry state.

9. The method of claim 1, wherein step (d) is carried out at a temperature of about 37° C.

10. The method of claim 1, wherein the blocker comprises 0.1-5% casein in buffer.

11. The method of claim 10, wherein the buffer is TBS.

12. The method of claim 1, wherein the secondary antibody is Anti-IgE-AP (alkaline phosphatase).

13. The method of claim 1, wherein the primary antibody is serum from a human subject.

14. The method of claim 1, wherein the thickness of the PVDF or other hydrophobic polymer layer is about 50-1000 μm.

15. The method of claim 1, wherein the thickness of the PVDF or other hydrophobic polymer layer is more than 1000 μm.

16. The method of claim 1 wherein the PVDF or other hydrophobic polymer layer is applied by lamination.

17. The method of claim 1, wherein the PVDF or other hydrophobic polymer layer is applied by a spray or spin coat.

18. The method of claim 1, wherein the rigid support is selected from the group consisting of a silanated material, glass and plastic.

19. The method of claim 1, wherein the rigid support is a glass or plastic microscope slide.

20. The method of claim 1, wherein the rigid support has a 3 dimensional surface that includes channels for sample processing.

21. The method of claim 1, wherein the substrate further comprises a bar code.

22. The method of claim 1, wherein the substrate further comprises a removable protective film.

23. The method of claim 1, wherein the substrate further comprises a template attached to the rigid support, wherein said template divides said PVDF substrate into at least two distinct sections, each section being configured to allow immobilization of a plurality of protein samples, and wherein said template is adapted to allow application of different chemical reagents to the at least two distinct sections of the PVDF substrate.

24. The method of claim 1, wherein phosphate-containing buffers and reagents are avoided.

25. The method of claim 1, wherein the level of the detection agent is determined using a developer comprising Nitro-Blue Tetrazolium Chloride (NBT), and 5-Bromo-4-Chloro-3′-Indolylphosphate I-Toluidine salt (BCIP).

26. A method of screening for an antigen comprising the steps of:
(a) preparing a PVDF or other 3-dimensional substrate for protein immobilization, comprising a rigid support and a PVDF or other hydrophobic polymer layer attached to said rigid support, wherein said PVDF or other hydrophobic polymer layer has a surface chemistry adapted to immobilize a protein sample and wherein said substrate is configured to allow immobilization of a plurality of samples on discrete addressable spots thereon;
(b) applying a first capture antibody to the PVDF or other 3-dimensional substrate to form a microarray;
(c) treating the microarray with a blocker;
(d) reacting the microarray with one or more capture proteins to form an antibody-antigen complex;
(e) reacting the microarray with a second capture antibody;
(f) reacting the microarray with a third antibody, wherein said third antibody is a detection agent; and
(g) determining a level of the detection agent and in turn determining the presence of the antigen.
